(19) United States
(12) Patent Application Publication
(10) Pub. No.: US 2025/0235332 A1
(43) Pub. Date: Jul. 24, 2025

(54) ARTICULATED ASSEMBLY

(71) Applicant: REVIVAL BIONICS, Venette (FR)

(72) Inventor: Guillaume Roger Albert Patrick BANIEL, Chantilly (FR)

(73) Assignee: REVIVAL BIONICS, Venette (FR)

(21) Appl. No.: 18/700,982

(22) PCT Filed: Oct. 10, 2022
(86) PCT No.: PCT/FR2022/051902

§ 371 Date: Apr. 12, 2024

(30) Foreign Application Priority Data
Oct. 13, 2021 (FR) 2110831

(51) Int. Cl. A61F 2/66; A61F 2/70

(52) U.S. Cl. CPC A61F 2/66; A61F 2/70; A61F 2002/6614; A61F 2002/701

(57) ABSTRACT

An articulated assembly including, an input element, an output element, a torsion bar capable of deforming about a longitudinal axis of the torsion bar, and an actuator. The input element is rotationally coupled to a first zone of the torsion bar and the output element is rotationally coupled to a second zone of the torsion bar, the input element further being connected to the actuator, the input element and the output element being guided in rotation relative to one another along an axis of the torsion bar.

### TECHNICAL FIELD

This disclosure relates to the field of articulated assemblies used in active or passive prostheses. More particularly, this disclosure concerns the field of foot prostheses.

### BACKGROUND

The goal of prostheses, active or passive, is to compensate for the disability of amputees by providing the function(s) of the amputated limb. For example, depending on the degree of amputation, a prosthetic foot potentially must carry out the function of the calf muscles, the foot dorsiflexion muscles, or the function relating to the Achilles tendon. In addition, prostheses, in particular active prostheses, must also integrate a controller and a battery in order to replace the control function normally provided by the brain and to compensate for the energy expenditure normally provided for by the metabolism.

Thus, active prostheses generally include an electric linear actuator composed of a motor-screw system. This actuator is generally placed in series with a spring element acting as an artificial Achilles tendon and making it possible to reduce the peak power to be provided by the actuator during use, the paired actuator/spring being known by the name “Series Elastic Actuator”.

Numerous solutions have been proposed by the prior art to meet the needs of people affected by amputation, in particular the need for prosthesis comfort, compactness, and autonomy of the prosthesis as well as its adaptability to the user's attributes. However, the solutions proposed in the prior art generally do not allow correctly addressing the needs cited above, and many drawbacks remain. For example, in the case of foot prostheses, the springs constituting the artificial Achilles tendon do not always have a stiffness specifically adapted to a patient or are difficult to adapt to the weight of each patient.

In addition, the architectural choices in the solutions of the prior art can limit the integration of all the components required to implement an active prosthesis (including for example a motor, electronics, a battery, an artificial Achilles tendon such as a spring) within a casing of a design reasonably identical to the volume of the absent limb, or can limit the integration of a number of battery cells compatible with an autonomy of a day or more, for the patients' benefit.

### SUMMARY

This disclosure improves the situation.

An articulated assembly is thus proposed which can comprise:
an input element,
an output element,
a torsion bar capable of deforming about a longitudinal axis of the torsion bar,
actuation means,

wherein the input element is rotationally coupled to a first zone of the torsion bar and the output element is rotationally coupled to a second zone of the torsion bar that is at a distance from said first zone, the input element further being connected to the actuation means, the input element and the output element being guided in rotation relative to one another along an axis of the torsion bar.

Advantageously, such an articulated assembly makes it possible to obtain a compact system for storing and supplying energy, by means of the torsion bar, in particular when it is used within a prosthetic or orthotic device. Such a structure makes it possible to reduce the overall dimensions in a manner that allows integrating other elements more easily, such as one or more batteries, circuits, one or more motors, etc.

The input element and output element may be respectively connected to the first zone and the second zone of the torsion bar via embedding connections.

The torsion bar may have a stiffness value of between 100 and 10,000 N.m per radian, preferably between 100 and 2,000 N.m per radian.

The torsion bar, the input element, and the output element may be configured so that the torsion bar is removable.

The torsion bar can thus easily be replaced when it is damaged or when the stiffness of the torsion bar needs to be adjusted. Advantageously, when the articulated assembly is integrated into a prosthesis (e.g. active), it is possible to adjust the stiffness of the torsion bar according to the patient's morphology or physiognomy. For example, it is possible to have classes of torsion bars, where each class is adapted to a patient's physiognomy, such as their weight.

The assembly may comprise a base that is hinged relative to the input element, the base supporting the actuation means.

The actuation means may comprise a linear actuator configured to move an actuating member capable of being moved along an actuation axis that is non-collinear, for example perpendicular, to the axis of the torsion bar, the movement of the actuating member causing rotation of the input element relative to the output element.

The linear actuator may be a hydraulic cylinder, a pneumatic cylinder, a screw-nut system driven by a motor, for example of the ball screw or planetary roller screw (also called standard roller screw) type, an electric motor, etc. Thus, according to one example, when the linear actuator is a screw-nut system, the actuating member may correspond to a screw set in motion by the nut driven by the motor. According to another example, when the linear actuator is a screw-nut system, the actuating member may correspond to a nut set in motion by a screw driven by the motor.

The screw-nut system driven by a motor may comprise a driving pulley coupled to the motor and a driven pulley coupled to the screw, the driving pulley and the driven pulley being connected by a drive belt.

The maximum power provided by the actuator may be between 50 and 1000 Watts, in the case of use in a prosthesis and during walking.

The total volume of the actuator and/or the total volume of the motor with a screw-nut system may be between 100 and 1500 cm3.

The actuating member is in a pivoting or ball joint connection with the input element.

The pivoting connection between the actuating member and the input element may be established through a bearing, for example a plain bearing, a ball, roller, or needle bearing, or a ball-and-socket fitting. The hinge pin may be fixed by shrink-on fittings or by elastic split rings.

The actuation means may be mounted directly or indirectly on or in the base. Indirect can be understood to mean that an additional element is arranged between the actuation means and the base.

Control means, such as a control unit, may be used to control the various elements of the prosthetic or orthotic device; these control means may be mounted on and/or in the base. The control means may also be mounted on the actuation assembly.

The linear actuator may be hinged to the base in a pivoting connection in which the pivot axis is parallel to the axis of the torsion bar and in which the torsion of the torsion bar or the rotation of the input element causes rotation of the linear actuator relative to the base about the pivot axis.

In one or more embodiments, at least one spring may be comprised between the linear actuator and the base; the at least one spring may be configured to compress or relax during relative movement between the linear actuator and said base.

Advantageously, it is then possible to adjust the overall stiffness value of the articulated assembly at the torsion bar. In one or more embodiments, the at least one spring may be configured so that the overall stiffness at the torsion bar may be between 100 and 10,000 N.m per radian.

Advantageously, by taking all the components into account when calculating the equivalent stiffness of the system on the axis of the torsion bar, this allows reducing the peak power in the motor.

Furthermore, the fact that several components contribute, in series, to the overall stiffness makes it possible to precisely adjust the stiffness of the torsion bar as well as its fatigue resistance.

In one or more embodiments, a first end of the torsion bar forms the first zone and a second end of the torsion bar forms the second zone, said torsion bar being configured to deform by torsion in a working portion that is between the first zone and the second zone, the input element and the output element respectively comprising a first tubular section and a second tubular section which are configured to fit into each other, together forming a tubular body housing the torsion bar, the first tubular section of the input element comprising a first internal coupling surface at a first end of the tubular body, configured to be rotationally coupled with the first zone of the torsion bar about the axis of the torsion bar, and the second tubular section of the output element comprising a second internal coupling surface at a second end of the tubular body, configured to be rotationally coupled with the second zone of the torsion bar about the axis of the torsion bar, and wherein the first tubular section and the second tubular section respectively comprise first guide surfaces, respectively internal and external to the first tubular section and to the second tubular section, or vice versa, the first guide surfaces being arranged in an intermediate position between the first end and the second end of the tubular body, the internal and external first guide surfaces being configured to ensure guidance in rotation of the output element relative to the input element about the axis of the torsion bar.

Second end of the tubular body can be understood to mean a second end opposite to the first end of the tubular body.

The guide surfaces may provide guidance by direct contact or may be implemented by means of a bearing, such as a ring, a bushing, or a rolling bearing for example.

The output element may comprise a second portion, distinct from the second tubular section, in pivoting connection with the first tubular section of the input element, via second guide surfaces which are respectively external and internal to the first tubular section and to the second portion, arranged at the first end of the tubular body.

The tubular body extending between the first end and the second end may entirely accommodate the torsion bar, the torsion bar preferably being of the same length as the tubular body along the axis of the torsion bar.

Thus, advantageously, it is possible to obtain a very compact energy charging and powering system, ideal for numerous devices such as large orthopedic devices (prostheses and orthotics), or even robotic devices (e.g. cobot or exoskeleton).

This disclosure also relates to a prosthetic device according to this disclosure; the prosthetic device may comprise:
the base is a tibial base intended to be fixed to a limb stump, for example by means of a socket, the tibial base carrying the actuation means,
a ground contact blade configured to pivot relative to the base about the axis of the torsion bar, and wherein the input element is connected to an output of the actuation means and the output element is connected to the ground contact blade,
the torsion bar being configured to store energy during walking by the torsion of the torsion bar, and to supply energy for propulsion simultaneously with the work from the actuation means.

Such a structure makes it possible to more faithfully reproduce the biomechanical characteristics of a human ankle and in particular its propulsive nature.

The tibial base may comprise a hollow body receiving the actuation means.

Advantageously, this base can protect the actuation means and be the focus of design work to improve the aesthetics of the system for the patients' benefit.

The hollow body of the tibial base may also carry control means such as a control unit which allows controlling the various elements of the prosthetic device.

The tibial base may have a longitudinal direction perpendicular to the torsion bar, and the actuation means comprise a motor and a screw-nut system, the screw-nut system and the motor being arranged so that they overlap along the longitudinal axis.

Advantageously, such an arrangement makes it possible to increase the compactness of the prosthetic device, to reduce its height in order to accommodate a larger number of patients of varying stump length and size, without sacrificing the performance required for the actuation means.

The screw-nut system driven by a motor may comprise a driving pulley coupled to the motor and a driven pulley coupled to the screw, the driving pulley and the driven pulley being connected by a drive belt.

A space may be provided between the torsion bar and the contact plate, for receiving an independent energy source, for example a removable one, for the actuation means.

Thus, advantageously, it is possible to reduce the overall dimensions of the prosthetic device by avoiding having the battery placed on the outside of the device. In addition, the battery can thus be concealed in a casing, for example an aesthetic casing in the shape of a foot intended to be worn in a shoe.

This disclosure also relates to a large orthopedic device comprising at least one articulated assembly according to this disclosure.

This disclosure also relates to an articulated system comprising at least one articulated assembly of the aforementioned type, wherein the articulated system comprises at least a first section and a second section which are hinged relative to each other, the first section forming the input element of the articulated assembly, the second section forming the output element of the articulated assembly.

### BRIEF DESCRIPTION OF DRAWINGS

Other features, details, and advantages will become apparent upon reading the detailed description below, and upon analyzing the appended drawings, in which:

FIG. 1a schematically illustrates a perspective view of the articulated assembly of this disclosure.

FIG. 1b schematically illustrates an exploded view of the articulated assembly of this disclosure.

FIG. 1c schematically illustrates a section view along plane ZY of the articulated assembly of this disclosure.

FIG. 2a schematically illustrates a perspective view of a prosthetic device such as a prosthetic foot comprising the articulated assembly of this disclosure.

FIG. 2b schematically illustrates a side view along plane ZX of a prosthetic device such as a prosthetic foot comprising the articulated assembly of this disclosure.

FIG. 2c schematically illustrates a section view along plane ZY of a prosthetic device such as a prosthetic foot comprising the articulated assembly of this disclosure.

### DETAILED DESCRIPTION

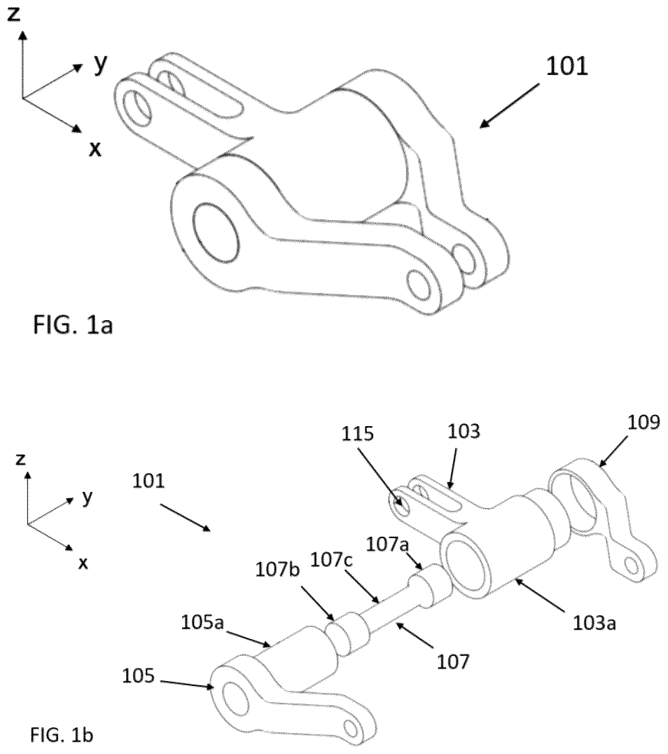

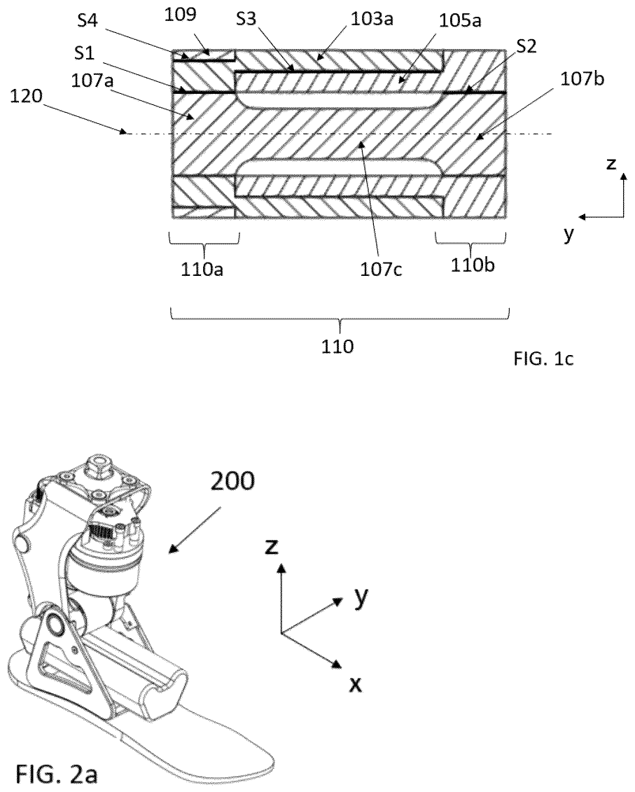

FIGS. 1a, 1b, and 1c schematically illustrate the articulated assembly in a perspective view, an exploded view, and a section view.

With reference to FIGS. 1a to 1c, articulated assembly 101 may comprise an input element 103, an output element 105, a torsion bar 107 capable of deforming around a longitudinal axis 120 of the torsion bar, and actuation means. Input element 103 may comprise a tubular section from which a connecting rod extends, for example forming a clevis joint. In particular, the free end of the arm may comprise an orifice intended for mounting a hinge.

The tubular section of input element 103 may be rotationally coupled to a first zone 107a of torsion bar 107, and output element 105 may be rotationally coupled to a second zone 107b of the torsion bar. Input element 103, in particular the arm, may be connected to the actuation means, and the input element and the output element 105 may be guided in rotation relative to one another along axis 120 of the torsion bar.

For example, torsion bar 107 may comprise a first end forming the first zone of the torsion bar and may comprise a second end forming the second zone of the torsion bar. The first zone and the second zone of the torsion bar may be cylindrical.

Torsion bar 107 may comprise a working portion 107c, for example cylindrical, between the first end and the second end of torsion bar 107, configured to deform by torsion. First zone 107a and second zone 107b of torsion bar 107 may have a larger cross-section or diameter than the cross-section or diameter of working portion 107c.

The stiffness of torsion bar 107 may be between 100 and 10,000 N.m per radian, enabling articulated assembly 101 to accommodate a large number of applications.

In one or more embodiments, working portion 107c may have a length of between 20 and 70 mm. The diameter of the working portion is for example between 5 and 15 mm.

Furthermore, input element 103 and output element 105 of articulated assembly 101 may respectively comprise a first tubular section 103a, for example cylindrical, and a second tubular section 105a, for example cylindrical, which are configured to fit into each other, together forming a tubular body 110 housing torsion bar 107. A connecting rod extends from tubular section 105a, the free end of said connecting rod comprising an orifice intended for the passage of a hinge pin, not shown.

First tubular section 103a of input element 103 may comprise a first internal coupling surface S1 at a first end 110a of the tubular body, configured to be rotationally coupled with first zone 107a of torsion bar 107, about axis 120 of torsion bar 107.

Similarly, second tubular section 105a of output element 105 may comprise a second internal coupling surface S2 at a second end 110b of tubular body 110, configured to be rotationally coupled with the second zone of torsion bar 107, about axis 120 of torsion bar 107.

For example, first surface S1 and second surface S2 may respectively be rotationally coupled with first zone 107a and second zone 107b via respective embedding connections. The embedding connections may be established by means of complementary grooves between first surface S1 and first zone 107a as well as between second surface S2 and second zone 107b. The embedding connections may be implemented by means of complementary polygonal shapes, for example square or hexagonal, between first surface S1 and first zone 107a as well as between second surface S2 and second zone 107b. Translational stop means between the parts concerned may be provided by any appropriate means, preferably removable.

The use of grooves and removable translation stop means can make it possible to have a removable torsion bar 107 which thus can be changed according to its wear, the physiognomy or morphology of the patient, or changes in the patient's physiognomy or morphology.

Rather than using grooves for the embedding connections, it may be possible to use any other type of transmission via obstructions between a shaft and a hub, for example a square, hexagonal, polygonal, spiked, serrated, conical or biconical assembly.

The length of each tubular section 103a, 105a may be between 20 and 70 mm. Similarly, the diameter of each tubular section may be between 10 and 40 mm.

The tubular body which extends between first end 110a and second end 110b may entirely accommodate torsion bar 107. The torsion bar may be of the same length as the tubular body along axis 120 of torsion bar 107.

In addition, first tubular section 103a and second tubular section 105a may respectively comprise first guide surfaces S3, respectively internal and external to first tubular section 103a and to second tubular section 105a, or vice versa. For example, the first surfaces may comprise the internal surface of first tubular section 103a and the external surface of second tubular section 105a which are in contact S3 to ensure rotational guidance of the output element relative to the input element about axis 120 of torsion bar 107.

Conversely, in one or more embodiments, first tubular section 103a may comprise an external surface in contact with an internal surface of second tubular section 105a so as to allow guided rotation of the input element relative to the output element.

These first guide surfaces may be arranged in an intermediate position between first end 110a and second end 110b of tubular body 110.

In addition, the output element may comprise a second portion 109, distinct from second tubular section 105a, comprising an annular zone in pivoting connection with first tubular section 103a of the input element. The pivoting connection may be implemented by second guide surfaces S4, respectively external and internal to first tubular section 103a and to the second portion, and which may be arranged at the first end of the tubular body. A small connector extends from the annular zone, the free end of the connector comprising an orifice intended for the passage of a hinge pin, not shown.

Input element 103, in particular the free end of the small connector of the input element, may be connected 115 to actuation means (not shown in FIGS. 1a-1c) by a pivoting connection.

The actuation means may comprise a linear actuator configured to move an actuating member capable of being moved along an axis 220 perpendicular to axis 120 of torsion bar 107.

By way of example, the linear actuator may be a jack screw, comprising a motor comprising an output shaft, the output shaft being driven in rotation and being coupled to a speed reducer, for example of the screw-nut type, the screw being coupled to the output shaft of the motor, possibly via a pulley system, or forming the output shaft of the motor. Rotation of the screw causes translational movement of the nut along the axis of screw 203. The nut is hinged relative to the free end of the connecting rod of the input element, by means of the axis XX.

The various elements comprised in the articulated assembly may be made of metal.

Such an articulated assembly can be used within a device such as an articulated system comprising one or more articulated assemblies, in large orthopedic devices (prostheses or orthotics, for example for the foot or knee), or in robotics, for example in cobots or exoskeletons.

For example, the articulated system may be a cobot such as an articulated arm used in cobotics, each articulation of the robotic arm possibly comprising one or more articulated assemblies according to the present description, arranged in series or in parallel.

According to another example, such an articulated assembly 101 may be integrated into an active prosthetic foot, articulated assembly 101 then fulfilling the function of the Achilles tendon.

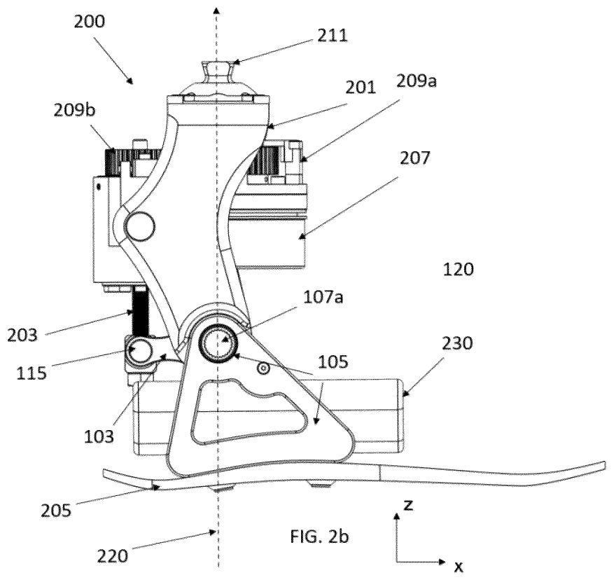

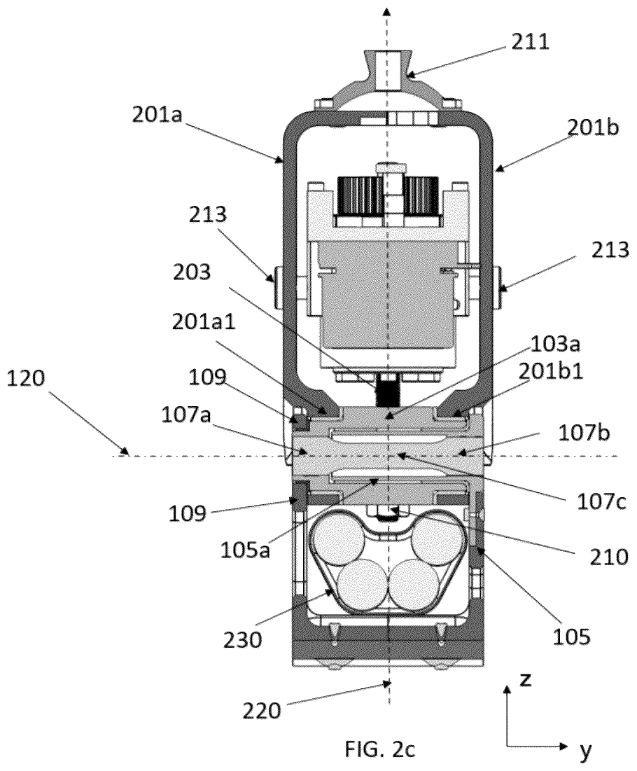

FIGS. 2a to 2c schematically illustrate a prosthetic device such as a prosthetic foot, comprising the articulated assembly of this disclosure.

FIG. 2a corresponds to a perspective view, FIG. 2b to a side view of the prosthetic foot along plane ZX, and FIG. 2c illustrates a section view ZY along a plane comprising the axis of torsion bar 107.

With reference to FIGS. 2a and 2b, prosthetic foot 200 may comprise the articulated assembly mounted in a tibial base 201 intended to be fixed to a stump by means of a socket. For example, tibial base 201 may be in the form of an inverted U-shaped support comprising a base at its top, from which two side walls extend downwards. A pyramidal adapter 211 extends upward from the base. Said adapter has for example a pyramid shape with a polygonal base, in particular a square base, allowing attachment to a person's stump by means of a socket.

Furthermore, output element 105 may be connected (e.g. directly or indirectly) to a ground contact blade 205 (i.e. equivalent to the arch of a foot) that is configured to pivot relative to the tibial base about torsion axis 120 when walking.

The ground contact blade may be a blade made of composite material, for example based on carbon fibers, glass, or Kevlar.

The ground contact blade may be configured so that its flexion contributes to the equivalent stiffness of the entire system on the axis of the torsion bar.

Each wall 201a; 201b of the tibial base may be pivotably mounted on tubular body 110 and between the two ends of tubular body 110a; 110b. More specifically, each wall 201a; 201b may have an orifice whose inside edge defines a cylindrical surface guided in rotation, for example by means of bush bearings or rings or rolling bearings, on complementary cylindrical surfaces formed by shoulders of first tubular section 103a of the input element. The base is thus in a pivoting connection with tubular body 110 which comprises the input element, the output element, and the torsion bar. Obviously, other types of connection can be considered.

In addition, tibial base 201 may delimit a hollow area, in which the actuation means are housed.

In one or more embodiments, control means such as a control unit which allows controlling the various elements of the prosthetic device may also be mounted on tibial base 201, for example in the hollow area.

Such an arrangement of the actuation means and/or control means in tibial base 201 allows increasing the compactness of the artificial foot, an important criterion for any wearer of an active prosthesis.

Furthermore, the linear actuator may be a motor 207 driving a screw-nut system as described above. Motor 207 may comprise a body and an output shaft intended to be driven in rotation, and coupled to a driving pulley 209a. The screw-nut system also comprises a body in which the nut is pivotably mounted, the nut being engaged on screw 203. The body is hinged on tibial base 201, at a hinge pin 213 perpendicular to the axis of the screw and parallel to axis 115 and to axis 107b. The body of the motor is fixed relative to the body of the screw-nut system. The nut is also coupled to a driven pulley 209b. Driving pulley 209a is capable of driving the driven pulley, via a belt for example (not shown).

The rotation of the shaft of motor 207 causes rotation of the nut of a planetary roller screw (also called standard roller screw) or a ball screw internal to the body, by means of pulleys 209a, 209b and the belt, and thus translation of the screw along the rotating nut, causing rotational movement of input element 103 relative to output element 105 by means of torsion bar 107.

In one or more embodiments, the pulley and belt system may be replaced by a gear system, for example straight spur gearing, bevel gearing, or even helical gearing. Alternatively, the motor shaft may directly drive the screw of the screw-nut system. According to yet another variant, the motor shaft may directly drive the nut of the screw-nut system.

In one or more embodiments, the total volume including the linear actuator and the paired driving/driven pulleys may be between 100 and 1500 cm3 for a maximum power of between 100 and 1500 Watts provided by the motor.

According to one or more examples, pivoting connection 115 between actuating member 203 and input element 103 may be established by means of a bearing, for example such as a plain bearing or a bearing with rolling elements such as a ball, roller, or needle bearing.

In order to allow rotation of input element 103 relative to tibial base 201 and/or to allow the torsion of torsion bar 107 when a wearer of the prosthetic foot is walking, the linear actuator pivots relative to base 201 around pivoting connection 213. The torsion of torsion bar 107 causes rotation of linear actuator 207 relative to the base, around pivoting connection 213. The torsion of torsion bar 107 allows energy to be stored which will then be delivered during the rest of the movement.

Power to the artificial foot, in particular to the actuation means, may be provided via an autonomous energy source 230 comprised in the prosthetic foot. In one or more embodiments, the autonomous energy source may be arranged within a space located between torsion bar 107 and the ground contact blade. Housing an independent energy source between torsion bar 107 and the contact blade is made possible by the compactness of the interlocking of the articulated assembly comprising the input element, torsion bar 107, and the output element connected to the contact blade.

In one or more embodiments, autonomous energy source 230 may be a battery, such as a lithium battery.

In one or more embodiments, the total volume of the battery may be between 100 and 300 cm3.

Such an arrangement within a prosthesis, such as a prosthetic foot, thus makes it possible to obtain an active prosthetic foot which is compact while being robust and which also provides sufficient autonomy for the daily routine of a person wearing a prosthetic foot.

In such an arrangement, the deformation of torsion bar 107 occurs during the ground contact phase, meaning when the tibia is passing over the foot, in the manner of the Achilles tendon. Torsion bar 107 then releases the stored energy, thus helping the motor to provide the torque required for propulsion in walking during the push phase.

More specifically, during the phase of ground contact by means of the ground contact blade, energy is stored by the torsion of torsion bar 107 via rotation of the input element relative to the output element. At the same time as the torsion (or deformation) of torsion bar 107, the motor drives the screw to enable the deformation of torsion bar 107. At the moment when the person wearing the prosthesis transfers his or her weight from one leg to the other, the motor pulls on the input element by raising the screw to trigger the propulsion, and due to the shifting of the weight, the stored energy can be released by releasing torsion bar 107 for the propulsion, simultaneously with the work from the motor.

Optionally, and in one or more embodiments, at least one spring, for example a tension spring, may be placed between the linear actuator and the base. The spring may be configured to extend or compress during the relative movement between the linear actuator and the base.

For example, the rotation of tibial base 201 about the pivot point, during walking, can exert traction on a spring located between tibial base 201 and the paired driving/driven pulleys. The use of this spring in series with torsion bar 107 makes it possible to vary the overall stiffness of the prosthetic device by adjusting the stiffness of the spring, the overall stiffness of the prosthetic device being on torsion bar 107.

In one or more embodiments, other configurations of the spring or of the location of the spring may be possible.

Furthermore, in general, in one or more embodiments, it is possible to improve the irreversibility of the planetary roller screw-nut assembly by modifying the slope of the threads, by making the thread asymmetrical, by alternating asymmetrical and symmetrical threads between screws, rollers, and nuts, or by adding gaskets to increase the friction.

1-20. (canceled)

21. An articulated assembly comprising:
an input element,
an output element,
a torsion bar capable of deforming about a longitudinal axis of the torsion bar,
actuation means,
wherein the input element is rotationally coupled to a first zone of the torsion bar and the output element is rotationally coupled to a second zone of the torsion bar, the input element further being connected to the actuation means, the input element and the output element being guided in rotation relative to one another along an axis of the torsion bar.

22. The articulated assembly according to claim 21, wherein the input element and the output element are respectively connected to the first zone and to the second zone of the torsion bar via embedding connections.

23. The articulated assembly according to claim 21, wherein the torsion bar has a stiffness value of between 100 and 10,000 Nm per radian.

24. The articulated assembly according to claim 21, wherein the torsion bar, the input element, and the output element are configured so that the torsion bar is removable.

25. The articulated assembly according to claim 21, wherein the assembly comprises a base that is hinged relative to the input element, the base supporting the actuation means.

26. The articulated assembly according to claim 25, wherein the actuation means comprise a linear actuator configured to move an actuating member capable of being moved along an actuation axis that is non-collinear, for example perpendicular, to the axis of the torsion bar, the movement of the actuating member causing rotation of the input element relative to the output element.

27. The articulated assembly according to claim 26, wherein the actuating member is in a pivoting or ball joint connection with the input element.

28. The articulated assembly according to claim 27, wherein the input element is connected to the base by a pivoting connection.

29. The articulated assembly according to claim 28, wherein the linear actuator is hinged to the base in a pivoting connection in which the pivot axis is parallel to the axis of the torsion bar and in which the torsion of the torsion bar or the rotation of the input element causes rotation of the linear actuator relative to the base about the second pivot axis.

30. The articulated assembly according to claim 29, comprising at least one spring between the linear actuator and the base, said at least one spring being configured to compress or relax during relative movement between the linear actuator and said base.

31. The articulated assembly according to claim 30, wherein the at least one spring is configured so that the overall stiffness is between 100 and 10,000 Nm per radian.

32. Articulated assembly according to claim 31, wherein a first end of the torsion bar forms the first zone and a second end of the torsion bar forms the second zone, said torsion bar being configured to deform by torsion in a working portion that is between the first zone and the second zone,
the input element and the output element respectively comprising a first tubular section and a second tubular section which are configured to fit into each other, together forming a tubular body housing the torsion bar, the first tubular section of the input element comprising a first internal coupling surface at a first end of the tubular body, configured to be rotationally coupled with the first zone of the torsion bar about the axis of the torsion bar, and the second tubular section of the output element comprising a second internal coupling surface at a second end of the tubular body, configured to be rotationally coupled with the second zone of the torsion bar about the axis of the torsion bar,
and wherein the first tubular section and the second tubular section respectively comprise first guide surfaces, respectively internal and external to the first tubular section and to the second tubular section, or vice versa, the first guide surfaces arranged in an intermediate position between the first end and the second end of the tubular body, the internal and external first guide surfaces configured to ensure guidance in rotation of the output element relative to the input element about the axis of the torsion bar.

33. The articulated assembly according to claim 32, wherein the output element comprises a second portion, distinct from the second tubular section, in pivoting connection with the first tubular section of the input element, via second guide surfaces which are respectively external and internal to the first tubular section and to the second portion, arranged at the first end of the tubular body

34. The articulated assembly according to claim 33, wherein the tubular body extending between the first end and the second end entirely houses the torsion bar, the torsion bar preferably being of the same length as the tubular body along the axis of the torsion bar.

35. A prosthetic device comprising an articulated assembly according to claim 25, comprising:
the base is a tibial base intended to be fixed to a limb stump, the tibial base carrying the actuation means,
a ground contact blade configured to pivot relative to the base about the axis of the torsion bar,
and wherein the input element is connected to an output of the actuation means and the output element is connected to the ground contact blade,
the torsion bar being configured to store energy during walking by the torsion of the torsion bar, and to supply energy for propulsion simultaneously with the work from the actuation means.

36. The prosthetic device according to claim 35, wherein the tibial base comprises a hollow body receiving the actuation means.

37. The prosthetic device according to claim 36, wherein the tibial base has a longitudinal direction perpendicular to the torsion bar, and the actuation means comprise a motor and a screw-nut system, the screw-nut system and the motor being arranged so that they overlap along the longitudinal axis.

38. The prosthetic device according to claim 35, wherein a space is present between the torsion bar and the contact plate, for receiving an independent energy source for the actuation means.

39. A large orthopedic device comprising at least one articulated assembly according to claim 21.

40. An articulated system comprising at least one articulated assembly according to claim 21, wherein the articulated system comprises at least a first section and a second section which are hinged relative to one another, the first section forming the input element of the articulated assembly, the second section forming the output element of the articulated assembly.
